(19) United States
(12) Patent Application Publication
(10) Pub. No.: US 2009/0088574 A1
(43) Pub. Date: Apr. 2, 2009

(54) CRYSTAL OF 1,2-DIHYDROPYRIDINE COMPOUND (TYPE IV)

(75) Inventors: Yoshio Urawa, Ibaraki (JP); Itaru Arimoto, Tokyo (JP)

Correspondence Address: BIRCH STEWART KOLASCH & BIRCH, PO BOX 747, FALLS CHURCH, VA, 22040-0747, US

(73) Assignee: EISAI R&D MANAGEMENT CO., LTD.

(21) Appl. No.: 12/158,294

(22) PCT Filed: Dec. 20, 2006
(86) PCT No.: PCT/JP2006/325394

§ 371 Date: Jul. 29, 2008

(30) Foreign Application Priority Data
Dec. 21, 2005 (JP) 2005-368426

(51) Int. Cl. C07D 213/22

(52) U.S. Cl. 546/257

(57) ABSTRACT

A crystal of 3-(2-cyanophenyl)-5-(2-pyridyl)-1-phenyl-1,2-dihydropyridin-2-one characterized in having a diffraction peak at a diffraction angle (2θ±0.2°) of 15.4° in a powder X-ray diffraction or others.

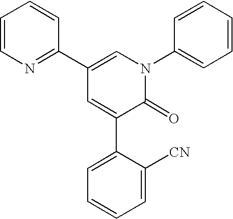

### TECHNICAL FIELD

This invention relates to crystals (type IV) of 1,2-dihydropyridine compound [3-(2-cyanophenyl)-5-(2-pyridyl)-1-phenyl-1,2-dihydropyridin-2-one] which are provided with antagonistic action against AMPA (alpha-amino-3-hydroxy-5-methyl-4-isoxazolepropionic acid) receptor and/or inhibitory action against kainate receptor and which are useful as a therapeutic or prophylactic agent for neurodegenerative diseases or others, as well as their production process.

### BACKGROUND ART

1,2-Dihydropyridine compounds possess antagonistic action against AMPA receptor and/or inhibitory action against kainate receptor and are useful as therapeutic or prophylactic agents for neurodegenerative diseases and others. Particularly, 3-(2-cyanophenyl)-5-(2-pyridyl)-1-phenyl-1,2-dihydropyridin-2-one (hereafter referred to as compound (1)) shows significant antagonistic action against AMPA receptor (see Patent Document 1).

Although Example 7 in Patent Document 1 discloses a process for producing the compound (1), there is merely described, “the residue is purified by silica gel column chromatography (ethyl acetate/hexane=1:2)” and there is no disclosure of the form of the obtained compound.

[Patent Document 1] WO/JP01/96308

### DISCLOSURE OF THE INVENTION

### Problems to be Solved by the Invention

The physical properties of medically useful form of compound such as crystal form or amorphous form affect medicament bioavailability, medicament substance purity, formulation preparations and the like. Thus, it is difficult to predict which crystal form or amorphous form of the compound is useful for pharmaceuticals in the development of pharmaceuticals. Therefore, there is a need for the finding of various crystal forms and amorphous forms useful as pharmaceuticals for each compound.

When a compound existing in crystal polymorphism is used as a medicament, it is necessary to stably supply the compound having uniform crystal form so that the uniform quality and the consistent potency required for the medicament may be guaranteed. There is also a need for the crystal form capable of maintaining the same quality during its storage and its formulation process (such as blending and granulation).

Since a drug substance is industrially used in a large amount, desirable crystal forms are those having low explosion concentration high limit and minimum ignition energy, index of explosiveness and dangerousness.

Generally, powders that tend to be charged have great adhesiveness to other objects; and there is concern about their adhesion to protective goods or the skin.

When a drug substance has chargeability, it happens that the production efficiency and workability lower if the compound adheres to a rotary blade at a milling stage in the manufacture of the compound, or adheres to and agglomerate on the production machines during the process of formulation. When a large quantity of powders having chargeability is processed on an industrial scale, there is the possibility that dust explosion will occur. It is, therefore, desired that a compound (crystal) having weak chargeability be used as the drug substance.

As for a compound having high pharmacological activity (such as the drug substance), the standpoint of the avoidance of exposure to the workers and the prevention of the facility contamination makes powders that do not tend to be charged desirable.

For the reasons above, when the active pharmaceutical ingredient of a medicament is obtained as a crystalline substance, it desirably comprises a homogeneous crystal form, has consistently preferable properties, and does not contain impurities such as metals. There has also been a need to develop a process for stably producing such crystals on an industrial scale.

Accordingly, it is an object of this invention to provide a crystal comprising a homogeneous crystal form of the compound (1) and a production process therefor.

### Means for Solving the Problems

As a result of the intensive and diligent studies, the present inventors discovered that a new homogeneous crystal form of the compound (1) could be obtained, upon which this invention has been completed.

Specifically, the present invention provides among others the following:

[1] A crystal of 3-(2-cyanophenyl)-5-(2-pyridyl)-1-phenyl-1,2-dihydropyridin-2-one having a diffraction peak at a diffraction angle (2θ±0.2°) of 15.4° in a powder X-ray diffraction.
[2] The crystal according to Item [1] further having diffraction peaks at diffraction angles (2θ±0.2°) of 16.6° and 24.3° in a powder X-ray diffraction.
[3] A medicament comprising the crystal according to Item [1].
[4] A pharmaceutical composition comprising the crystal according to Item [1].
[5] A therapeutic or prophylactic agent for an acute neurodegenerative disease comprising the crystal according to Item [1].
[6] A therapeutic or prophylactic agent for neuropathy caused by acute phase of cerebrovascular disorder, head injury, spinal cord injury or hypoxia, or neuropathy caused by hypoglycemia, comprising the crystal according to Item [1].
[7] A therapeutic or prophylactic agent for a chronic neurodegenerative disease comprising the crystal according to Item [1].
[8] A therapeutic or prophylactic agent for Alzheimer's disease, Parkinson's disease, Huntington's chorea, amyotrophic lateral sclerosis or spinocerebellar degeneration, comprising the crystal according to Item [1].
[9] A therapeutic or prophylactic agent for epilepsy, hepatic encephalopathy, peripheral neuropathy, Parkinsonism, spastic paralysis, pain, neuralgia, schizophrenia, anxiety, drug-dependence, nausea, vomiting, dysuria, vision impairment caused by glaucoma, hearing impairment caused by antibiotics, or food poisoning, the agent comprising the crystal according to Item [1].
[10] A therapeutic or prophylactic agent for infectious encephalomyelitis, cerebrovascular dementia, or dementia or neurological symptom caused by meningitis, comprising the crystal according to Item [1].
[11] A therapeutic or prophylactic agent for a demyelinating disease comprising the crystal according to Item [1].
[12] The therapeutic or prophylactic agent according to Item [10], wherein the infectious encephalomyelitis is HIV encephalomyelitis.
[13] The therapeutic or prophylactic agent according to Item [11], wherein the demyelinating disease is encephalitis, acute sporadic encephalomyelitis, multiple sclerosis, acute polyradiculoneuropathy, Guillain-Barre syndrome, chronic inflammatory demyelinating polyradiculoneuropathy, Marchifava-Bignami disease, central pontomedullary myelinolysis, neuromyelitis optica, Devic's disease, Balo's disease, HIV-associated myelopathy, HTLV-associated myelopathy, progressive multifocal leukoencephalitis or a secondary demyelinating disease.
[14] The therapeutic or prophylactic agent according to Item [13], wherein the secondary demyclinating disease is CNS lupus erythematosus, polyarteritis nodosa, Sjoegren's syndrome, sarcoidosis or dissociated cerebral vasculitis.

According to the invention, it has become possible that the compound (1) is readily produced as a homogeneous crystal form. The crystals of the invention have preferable properties and are suitable for use as an active ingredient of therapeutic or prophylactic agents for neurodegenerative diseases or others.

### BRIEF DESCRIPTION OF THE DRAWINGS

FIG. 1 shows a powder X-ray diffraction pattern of the crystals obtained in Reference Example A1.

FIG. 2 shows a powder X-ray diffraction pattern of the crystals obtained in Example 1.

FIG. 3 shows a powder X-ray diffraction pattern of Anhydrous form II before and after mixing operation.

FIG. 4 shows a powder X-ray diffraction pattern of Crystal form IV before and after mixing operation.

### BEST MODES FOR CARRYING OUT THE INVENTION

This invention will be described in detail hereafter.

The crystals of the invention are crystals of the compound (1) with the characteristics described below. (Although the respective measurement conditions for powder X-ray diffraction patterns are not particularly limited, the measurement should preferably be conducted under the measurement conditions for the powder X-ray diffraction patterns as will be described in Examples below.)

(1) A crystal having a diffraction peak at a diffraction angle (2θ±0.2°) of 15.4° in a powder X-ray diffraction;
(2) A crystal having a diffraction peak at a diffraction angle (2θ±0.2°) of 15.4°, 16.6° and 24.3° in a powder X-ray diffraction.

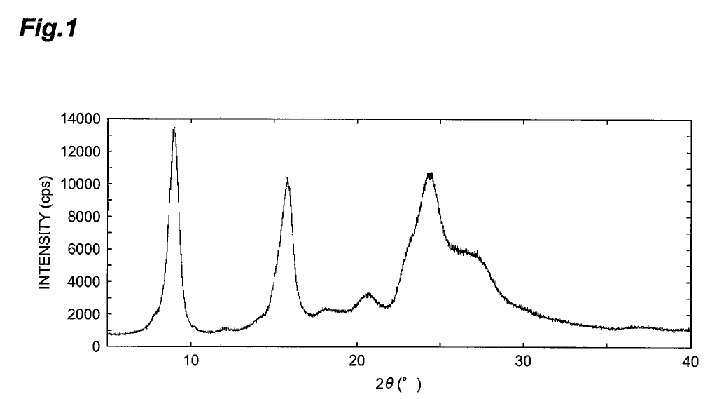

These characteristic peaks in the powder X-ray diffraction are not observable in the crystals obtained by the production process disclosed in Patent Document 1 (see Reference Example A1, Table 1 and FIG. 1 as described below).

As for a diffraction angle (2θ) in the powder X-ray diffraction analysis, errors in the diffraction angle, generally, may occur within the range of ±0.2°. It is, therefore, to be understood that the values of the diffraction angles may include numerals on the order of ±0.2°. Accordingly, this invention encompasses not only crystals having completely matching diffraction angles of the peaks in powder X-ray diffraction, but also crystals having matching diffraction angles of the peaks within the errors of about ±0.2°.

In this Specification, “having a diffraction peak at a diffraction angle (2θ±0.2°) of 15.4°” means “having a diffraction peak at a diffraction angle (2θ) of 15.2° to 15.6°.” The term “having a diffraction peak at a diffraction angle (2θ±0.2°) of 16.6°” means “having a diffraction peak at a diffraction angle (2θ) of 16.4° to 16.8°.” The term “having a diffraction peak at a diffraction angle (2θ±0.2°) of 24.3°” means “having a diffraction peak at a diffraction angle (2θ) of 24.1° to 24.5°.”

[General Production Method of Crystal Form IV of Compound (1)]

Crystals of the present invention can be produced from a raw material of the compound (1) which is synthesized according to a method as described in the Example 7 of above-mentioned Patent Document 1 (WO/JP01/96308) or Production Example 4 shown below. Crystals of the compound (1) of the present invention can be stably synthesized by treating the compound (1) in the same matter of Example 1 or 2 shown below. Compound (1) used as a raw material can be in any form, i.e. can be hydrate or anhydrous form, amorphous or crystalline form (including plural crystal polymorphs), or a mixture thereof.

The use of the compound (1) as a therapeutic agent for neurodegenerative diseases or others is fully disclosed in Patent Document 1. The crystals of the present invention can be used as the active ingredient in the therapeutic agent for neurodegenerative diseases or others. The entire disclosure of Patent Document 1 is thus hereby incorporated in this Specification by reference.

When the compound of this invention is to be used as a medicament, it is normally compounded with suitable pharmaceutical ingredients to prepare pharmaceutical products for use. Notwithstanding, the use of a drug substance form of the compound of the present invention as a medicament should not be negated.

The pharmaceutical ingredients may include excipients, binders, lubricants, disintegrating agents, coloring agents, taste correctives, emulsifiers, surfactants, dissolving aids, suspending agents, isotonizing agents, buffering agents, preservatives, antioxidants, stabilizers, absorption enhancers, and the like, all of which are generally used in medicaments. If desired, these agents may be combined for use.

The excipients may include, for example, lactose, white soft sugar, glucose, corn starch, mannitol, sorbitol, starch, alpha starch, dextrin, crystalline cellulose, light silicic anhydride, aluminum silicate, calcium silicate, magnesium aluminometasilicate, calcium hydrogenphosphate, and the like.

The binders may include, for example, polyvinyl alcohol, methylcellulose, ethylcellulose, gum Arabic, tragacanth, gelatin, shellac, hydroxypropylmethylcellulose, hydroxypropylcellulose, carboxymethylcellulose sodium, polyvinylpyrrolidone, macrogol, and the like.

The lubricants may include, for example, magnesium stearate, calcium stearate, sodium stearyl fumarate, talc, polyethylene glycol, colloidal silica, and the like.

The disintegrating agents may include, for example, crystalline cellulose, agar, gelatin, calcium carbonate, sodium hydrogencarbonate, calcium citrate, dextrin, pectin, low-substituted hydroxypropylcellulose, carboxymethylcellulose, carboxymethylcellulose calcium, croscarmellose sodium, carboxymethyl starch, carboxymethyl starch sodium, and the like. The coloring agents may include iron sesquioxide, yellow iron sesquioxide, carmine, caramel, beta-carotene, titanium oxide, talc, riboflavin sodium phosphate, yellow aluminum lake, and the like, which have been approved as additives for medicaments.

The taste correctives agents may include cocoa powder, menthol, aromatic powder, mentha oil, borneol, powdered cinnamon bark, and the like

The emulsifiers or the surfactants may include stearyl triethanolamine, sodium lauryl sulfate, lauryl aminopropionic acid, lecithin, glycerin monostearate, sucrose fatty acid ester, glycerin fatty acid ester, and the like.

The dissolving aids may include polyethylene glycol, propylene glycol, benzyl benzoate, ethanol, cholesterol, triethanolamine, sodium carbonate, sodium citrate, Polysorbate 80, nicotinamide, and the like. The suspending agents may include, in addition to the surfactants, hydrophilic polymers such as polyvinyl alcohol, polyvinylpyrrolidone, methylcellulose, hydroxymethylcellulose, hydroxyethylcellulose, and hydroxypropylcellulose.

The isotonizing agents may include glucose, sodium chloride, mannitol, sorbitol and the like.

The buffering agents may include the buffers of phosphate, acetate, carbonate, citrate and the like.

The preservatives may include methylparaben, propylparaben, chlorobutanol, benzyl alcohol, phenethyl alcohol, dehydroacetic acid, sorbic acid and the like.

The antioxidants may include sulfite, ascorbic acid, alpha-tocopherol and the like.

The stabilizers may include those generally used in medicaments.

The absorption enhancers may include those generally used in medicaments.

The pharmaceutical products described above may include: oral agents such as tablets, powders, granules, capsules, syrups, troches, and inhalations; external preparations such as suppositories, ointments, ophthalmic ointments, tapes, ophthalmic solutions, nasal drops, ear drops, poultices, and lotions; and injections.

The oral agents may appropriately be combined with the auxiliaries described above to form preparations. In addition, the surfaces of the agents may be coated if necessary.

The external preparations may appropriately be combined with the auxiliaries, in particular, excipients, binders, taste correctives, emulsifiers, surfactants, dissolving aids, suspending agents, isotonizing agents, preservatives, antioxidants, stabilizers, or absorption enhancers to form the preparations.

The injections may appropriately be combined with the auxiliaries, in particular, emulsifiers, surfactants, dissolving aids, suspending agents, isotonizing agents, preservatives, antioxidants, stabilizers, or absorption enhancers to form the preparations.

When the compound of this invention is to be used as a medicament, its dosage level may differ depending on the symptoms, ages or others.

The compound is normally given in a single administration or in divided administrations 2 to 6 times daily at the following doses: from 0.05 to 10 mg (preferably from 0.1 to 5 mg) in the case of an oral agent; from 0.01 to 10 mg (preferably from 0.05 to 5 mg) in the case of an external preparation; and 0.01 to 5 mg in the case of an injection. Here, the actual amounts to be administered are indicated with respect to the oral agent and the injection, while the amount to be absorbed by the body is indicated with respect to the external preparation.

### EXAMPLES

This invention will be specifically described in detail by way of the following examples; however, the present invention is not to be limited to these examples.

### Production Example 1

### Synthesis of 5-(2-pyridyl)-1-phenyl-1,2-dihydropyridin-2-one

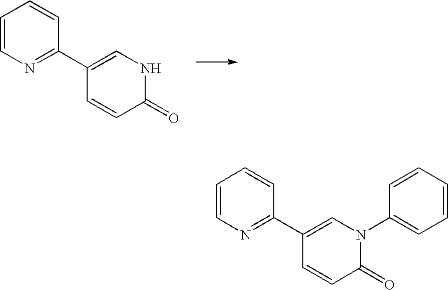

After a reactor was purged with nitrogen, a mixture of 5-(2-pyridyl)-1,2-dihydropyridin-2-one (7.33 kg: WO2004/009553), triphenylboroxine (9.0 kg), copper acetate (anhydrous) (0.80 kg), water (0.50 kg), pyridine (7.1 kg), and N,N-dimethylformamide (66.7 kg) was stirred in the reactor at an internal temperature of 28° C. for 1 hour.

While the air of which oxygen concentration was adjusted to 9% with nitrogen was blown into the reactor at a rate of 30 L/min, the reaction mixture was stirred at 39-40° C. (internal temperature) for 16 hours to yield reaction mixture 1A.

Water (191 kg) and 25% aqueous ammonia (85.8 kg) were charged in a separate reactor and cooled with cold water to 8.7° C. The reaction mixture 1A was then added to the reactor over 3 minutes. The reaction mixture was stirred for 4 hours at cooling with cold water. The precipitates in the reaction mixture were collected by filtration with a centrifuge and the filtrated residue was washed with 65 kg of water.

The precipitates, water (97 kg), and 25% aqueous ammonia (43.5 kg) were poured in a reactor and stirred for 1 hour while the temperature was maintained with warm water (25° C.). The precipitates in the reaction mixture were collected by filtration with a centrifuge and the filtrated residue was washed with 32.6 kg of water. The precipitates were then dried under reduced pressure (60° C.; 18 hours) to give 9.6 kg of 5-(2-pyridyl)-1-phenyl-1,2-dihydropyridin-2-one.

¹H NMR (400 MHz DMSO-d₆): δ 8.61-8.50 (m, 1H), 8.36 (d, 1H), 8.29 (dd, 1H), 7.90 (d, 1H), 7.80 (ddd, 1H), 7.56-7.45 (m, 5H), 7.27 (dd, 1H), 6.62 (d, 1H).

### Production Example 2

### Synthesis of 3-bromo-5-(2-pyridyl)-1-phenyl-1,2-dihydro-pyridin-2-one

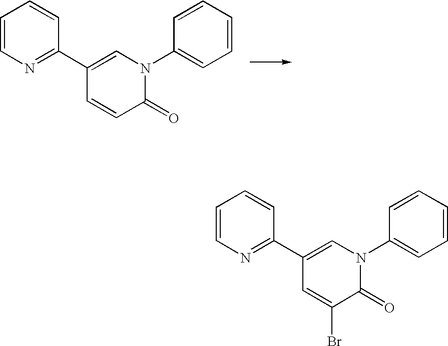

5-(2-Pyridyl)-1-phenyl-1,2-dihydropyridin-2-one (200 g), N-bromosuccinimide (157.7 g), and ethyl acetate (4 L) were added to a 10 L-reactor and the reaction mixture was stirred under a nitrogen stream at 30° C. (external temperature) for 9 hours and 20 minutes. 3% Hydrosulfite solution (2 L) and toluene (2 L) were added to the reaction mixture and then it was stirred at 55° C. (external temperature) for 30 minutes. After the completion of reaction, the aqueous layer (lower layer) in the reaction mixture was separated, and then, the organic layer was washed with water (2 L) four times. The solvent was evaporated at stirring under reduced pressure.

Subsequently, further addition of 1,2-dimethoxyethane (4 L) and concentration under reduced pressure gave a crude product of 3-bromo-5-(2-pyridyl)-1-phenyl-1,2-dihydropyridin-2-one.

### Production Example 3

### Synthesis of 3-(2-cyanophenyl)-5-(2-pyridyl)-1-phenyl-1,2-dihydropyridin-2-one

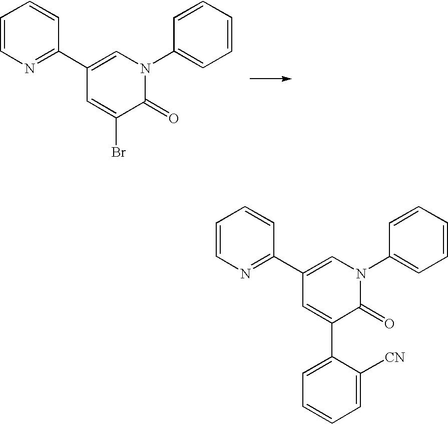

To the reactor containing the whole amount of the crude product of 3-bromo-5-(2-pyridyl)-1-phenyl-1,2-dihydropyridin-2-one obtained as the residue after concentration in Production Example 2 were added 2-(1,3,2-dioxaborinan-2-yl)benzonitrile (214.9 g), palladium acetate (3.44 g), triphenylphosphine (16.07 g), cuprous iodide (7.29 g), 1,2-dimethoxyethane (3.1 L) and potassium carbonate (158.8 g). Stirring at heating was carried out at 70° C. (external temperature) under a nitrogen atmosphere for 30 minutes and, then, at heating under reflux for 4 hours.

Subsequently, ethyl acetate (2.5 L) was added to the reaction mixture at 70° C. (external temperature) and the mixture was stirred for 10 minutes. The reaction mixture was filtrated and the filtrated residue was washed with ethyl acetate (2.5 L). This whole filtrate was transferred to a reactor, to which 12.5% aqueous ammonia (5 L) was further added. Stirring was carried out at 60° C. (external temperature) for 53 minutes. The lower layer (aqueous layer) in the reaction mixture was separated. 5% Brine (2.5 L) and 25% aqueous ammonia (2.5 L) were added to the remaining organic layer. After stirring, the lower (aqueous layer) was separated. 5% Brine (5 L) was further added to the remaining organic layer. After stirring, the lower (aqueous layer) was separated. The remaining organic layer was concentrated under reduced pressure, and then, acetone (4 L) was added, followed by concentration under reduced pressure.

Acetone (7.2 L) and water (0.8 L) were added to this residue, and it was dissolved by stirring at 60° C. (external temperature) for 1 hour and 10 minutes. Next, cooling was carried out at 38° C. (external temperature) for 18 minutes while stirring. To the reaction mixture was added 1 g of seed crystals, crystals of 3-(2-cyanophenyl)-5-(2-pyridyl)-1-phenyl-1,2-dihydropyridin-2-one hydrate. Stirring was carried out at 35° C. (external temperature) for 30 minutes. Subsequently, the reaction mixture was stirred at an external temperature being lowered by 5° C. every 30 minutes, and stirred at an external temperature of 10° C. for 17 hours.

Water (2.29 L) was added dropwise to the reaction mixture at stirring over a period of 3 hours and 10 minutes. After the addition, stirring continued for additional 1 hour and 20 minutes. The reaction mixture was filtrated and the filtrated residue was washed with 2 L of 50% acetone-water to give 3-(2-cyanophenyl)-5-(2-pyridyl)-1-phenyl-1,2-dihydropyridin-2-one (526.28 g) as a wet cake, which corresponded to 168.3 g as dry weight.

### Production Example 4

### 3-(2-Cyano-phenyl)-5-(2-pyridyl)-1-phenyl-1,2-dihydropyridin-2-one

### (Hydrate Crystal)

A 10 L-flask was charged with 526.28 g of 3-(2-cyanophenyl)-5-(2-pyridyl)-1-phenyl-1,2-dihydropyridin-2-one obtained as the wet cake in Production Example 3. Out of an acetone-water prepared from 5890 mL of acetone and 490 mL of water, 5.5 L was added to the flask and heated. Filtration was carried out after dissolution. While the 10-L flask and the filtrated residue were washed with the remaining total of the acetone water, all the filtrate was transferred to a 10-L flask.

The mixture was stirred at an external temperature of 40° C., and after the internal temperature reached 40° C., the external temperature was adjusted to 35° C. Next, 842 mg of 3-(2cyanophenyl)-5-(2-pyridyl)-1-phenyl-1,2-dihydropyridin-2-one hydrate was added to the mixture. After stirring the mixture for 30 minutes, the external temperature was changed to 30° C., and then to 25° C. after 30 minutes. The external temperature was lowered by 5° C. every 30 minutes thereafter to as low as 15° C. After stirring the mixture at an external temperature of 15° C. for 30 minutes, the external temperature was further lowered to 8° C. and stirring continued for 1 hour.

To the mixture was added dropwise 842 mL of water at 11° C. (internal temperature) over a period of 1 hour and 10 minutes. One hour after the completion of addition, the external temperature was changed to 0° C. and the mixture was stirred for 40 minutes. The external temperature was then lowered to −20° C. and stirring continued for 15 hours.

The precipitates in the mixture were collected by filtration. After the precipitates were washed with 1700 mL of 50% acetone-water, they were dried under aeration for 50 minutes. Subsequently, these precipitates were dried with a vibration drier at 40° C. under reduced pressure for 11 hours and were additionally dried at 60° C. for 3 hours.

After the temperature of the drier was cooled to room temperature, the external atmosphere was aspirated into the drier at 950 hpa for 4 hours to give 172.4 g of 3-(2cyanophenyl)-5-(2-pyridyl)-1-phenyl-1,2-dihydropyridin-2-one (crystal form of the hydrate).

¹H NMR (400 MHz, DMSO-d₆): δ 8.61-8.57 (m, 1H), 8.53-8.52 (d-like, 1H), 8.47 (d, 1H), 8.01 (d, 1H), 7.92 (d, 1H), 7.86-7.81 (t-like, 1H), 7.79-7.76 (t-like, 1H), 7.72 (d, 1H), 7.61-7.48 (m, 6H), 7.31-7.28 (m, 1H).

### Reference Example A1

### Production of anhydrous crystals of 3-(2-cyanophenyl)-5-(2-pyridyl)-1-phenyl-1,2-dihydropyridin-2-one

### (Anhydrous Form II)

In the same manner as the procedure after reaction work-up that are described in Example 7 in WO01/96308, the production was carried out below. The synthetic method for 3-(2-cyanophenyl)-5-(2-pyridyl)-1-phenyl-1,2-dihydropyridin-2-one [alternative name: 2-(2-oxo-1-phenyl-5-(pyridin-2-yl)-1,2-dihydropyridin-3-yl)benzonitrile] is described in Example 7 in WO01/96308 as well as in Production Example 3 above.

Ethyl acetate (400 mL) was added to 3-(2-cyanophenyl)-5-(2-pyridyl)-1-phenyl-1,2-dihydropyridin-2-one (8 g). The mixture was heated at 60° C. in a warm bath. Additional acetate (160 mL) was added to the mixture and the solids were dissolved by heating at 70° C. in the warm bath. After n-hexane (80 mL) was added to this solution, the solvent was evaporated under reduced pressure to give 7.7 g of a pale yellow powder.

¹H NMR (400 MHz, DMSO-d₆): δ 8.59-8.57 (m, 1H), 8.53 (d, 1H), 8.47 (d, 1H), 8.01 (d, 1H), 7.92 (d, 1H), 7.83 (ddd, 1H), 7.80-7.76 (m, 1H), 7.73-7.71 (d-like, 1H), 7.61-7.48 (m, 6H), 7.30 (dd, 1H).

### Example 1

### 3-(2-Cyanophenyl)-5-(2-pyridyl)-1-phenyl-1,2-dihydopyridin-2-one

### (Crystal Form IV)

A 200 mL flask was charged with 10 g of 3-(2-cyanophenyl)-5-(2-pyridyl)-1-phenyl-1,2-dihydropyridin-2-one (hydrate crystal), 100 mL of acetone and 2.5 mL of water, and the mixture was stirred at room temperature for 5 minutes. 30 mL of this slurry mixture was transferred to a test tube and further stirred at 0° C. for 2 hours. To this mixture, 5 mL of acetone/water mixed solvent prepared from 100 mL of acetone and 2.5 mL of water (hereinafter simply referred to as “acetone/water mixed solvent”) was added, and further stirred at 0° C. for 16 hours. This mixture was filtered to give 315 mg of 3-(2-Cyanophenyl)-5-(2-pyridyl)-1-phenyl-1,2-dihydropyridin-2-one (Crystal form IV).

### Example 2

### 3-(2-Cyanophenyl)-5-(2-pyridyl)-1-phenyl-1,2-dihydropyridin-2-one

### (Crystal Form IV)

A 200 mL 4-necked flask was charged with 3-(2-cyanophenyl)-5-(2-pyridyl)-1-phenyl-1,2-dihydropyridin-2-one (10.00 g). Acetone (100 mL) and water (2.5 mL) were added to the flask and the mixture was stirred at room temperature for 5 minutes. 30 mL of this slurry mixture was transferred to a 100 mL test tube and stirred at −5° C., followed by further adding 10 mL of acetone/water mixed solvent and stirring. A 5 mL sample was taken from this mixture, stirred for 2 hours, and 5 mL of acetone/water mixed solvent was added. This mixture was further stirred for 29 hours, followed by filtering the suspension. The obtained precipitate was dried under reduced pressure at room temperature to give 1.037 g of 3-(2-Cyanophenyl)-5-(2-pyridyl)-1-phenyl-1,2-dihydropyridin-2-one (Crystal form IV).

Measurement of Powder X-Ray Diffraction Pattern

The powder X-ray diffraction of the crystals obtained in each Reference Example and Examples were measured under the following measurement conditions according to the powder X-ray diffraction measurement method described in General Tests, Japanese Pharmacopeia.

### (Equipment)

Powder X-ray diffraction measurement apparatus: RINT-2000 (from Rigaku Corporation)

### (Operation Procedure)

A sample was ground using an agate mortar, taken on a glass plate having a diameter of 13 mm, and measured under the following conditions.

X-ray used: CuKα ray
Tube voltage: 40 kV
Tube current: 200 mA
Divergence slit: ½ deg
Receiving slit: 0.3 mm
Scattering slit: ½ deg
Scanning rate: 1 or 2°/min.
Scanning step: 0.01 or 0.02°
Measurement range (2θ): 5 to 40°

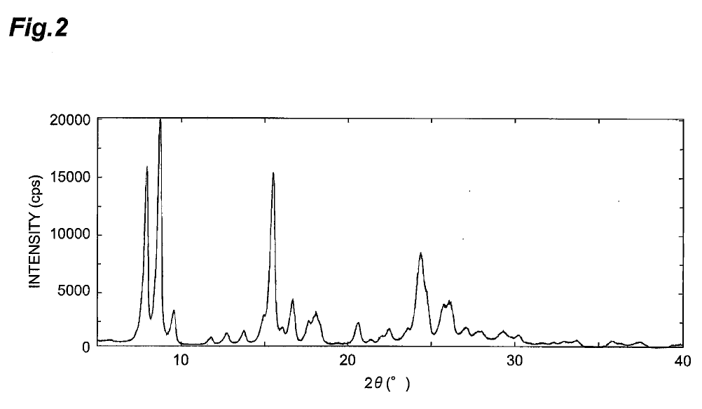

The powder X-ray diffraction pattern of the crystals obtained in Reference Example A1 is shown in FIG. 1, and the powder X-ray diffraction pattern of the crystals obtained in Example 1 is shown in FIG. 2.

Table 1 shows the peaks and their intensities at diffraction angles (2θ) for the crystals obtained in Reference Example A1, and Table 2 shows the peaks and their intensities at diffraction angles (2θ) for the crystals obtained in Example 1.

Based on FIG. 2 and Table 2 that represents the powder X-ray diffraction pattern of the crystals obtained in Example 1, it can be found that the powder X-ray diffraction pattern of the crystals obtained in Example 1 provides a characteristic peaks having a diffraction angle (2θ) of about 15.4°, about 16.6° and about 24.3°. Furthermore, it is confirmed that the powder X-ray diffraction pattern of the crystals obtained in Example 2 provides similar characteristic peaks.

FIG. 1 and Table 1 that represents the powder X-ray diffraction pattern of the crystals obtained in Reference Example A1, not having a diffraction angle (2θ) of about 15.4°, about 16.6°, suggest that crystals obtained in Reference Example A1 do not contain the same form as do the crystals obtained in Example 1.

TABLE 1

| PEAK | | HALF | | | RELATIVE |
|---|---|---|---|---|---|
| NUMBER | 2θ | WIDTH | D-VALUE | INTENSITY | INTENSITY |
| 1 | 9.010 | 0.588 | 9.8067 | 13370 | 100 |
| 2 | 15.850 | 0.682 | 5.5867 | 10137 | 76 |
| 3 | 24.390 | 0.847 | 3.6465 | 10672 | 80 |

TABLE 2

| PEAK | | HALF | | | RELATIVE |
|---|---|---|---|---|---|
| NUMBER | 2θ | WIDTH | D-VALUE | INTENSITY | INTENSITY |
| 1 | 7.890 | 0.318 | 11.1961 | 15360 | 77 |
| 2 | 8.680 | 0.329 | 10.1788 | 19905 | 100 |
| 3 | 9.550 | 0.341 | 9.2534 | 3392 | 17 |
| 4 | 11.810 | 0.341 | 7.4872 | 1125 | 6 |
| 5 | 12.740 | 0.365 | 6.9427 | 1438 | 7 |
| 6 | 13.730 | 0.341 | 6.4442 | 1658 | 8 |
| 7 | 15.410 | 0.353 | 5.7453 | 15360 | 77 |
| 8 | 16.630 | 0.376 | 5.3264 | 4355 | 22 |
| 9 | 17.620 | 0.294 | 5.0293 | 2548 | 13 |
| 10 | 18.070 | 0.600 | 4.9051 | 3017 | 15 |
| 11 | 20.600 | 0.412 | 4.3080 | 2267 | 11 |
| 12 | 21.390 | 0.329 | 4.1506 | 837 | 4 |
| 13 | 22.020 | 0.271 | 4.0333 | 1187 | 6 |
| 14 | 22.460 | 0.365 | 3.9553 | 1880 | 9 |
| 15 | 23.540 | 0.282 | 3.7702 | 1873 | 9 |
| 16 | 24.310 | 0.482 | 3.6583 | 8507 | 43 |
| 17 | 25.700 | 0.365 | 3.4635 | 3812 | 19 |
| 18 | 26.120 | 0.412 | 3.4088 | 4198 | 21 |
| 19 | 27.100 | 0.447 | 3.2877 | 1973 | 10 |
| 20 | 27.960 | 0.694 | 3.1885 | 1563 | 8 |
| 21 | 29.310 | 0.553 | 3.0446 | 1703 | 9 |
| 22 | 30.240 | 0.388 | 2.9531 | 1307 | 7 |
| 23 | 32.930 | 0.388 | 2.7177 | 845 | 4 |
| 24 | 33.690 | 0.424 | 2.6581 | 897 | 5 |
| 25 | 35.840 | 0.447 | 2.5034 | 843 | 4 |
| 26 | 37.480 | 0.518 | 2.3976 | 698 | 4 |

Physical Stability in Mixing Operation in the Presence of Water or Water-Ethanol (1:1) Mixed Solution

### (Operation Procedure)

About 60 to 150 mg of crystals obtained in Reference Example A1 (Anhydrous form II) and crystals obtained in Example 1 (Crystal form IV) were placed in an agate mortar, and the mixing operation was carried out at room temperature for a few minutes while the dropwise addition of water (or water-ethanol (1:1) mixed solution) continues. Subsequently, the crystals were dried at about 60° C. for 2 to 3 hours. The powder X-ray diffraction of the crystals obtained and crystals of before mixing operation were each measured under the following measurement conditions.

### (Equipment)

D8 DISCOVER with GADDS manufactured by Bruker AXS Ltd.

### (Condition)

### X-ray in use: CuKα ray

Tube voltage: 40 kV
Tube current: 200 mA
Scanning range (2θ): 5 to 40°
Collimator: 0.3 mm double slit

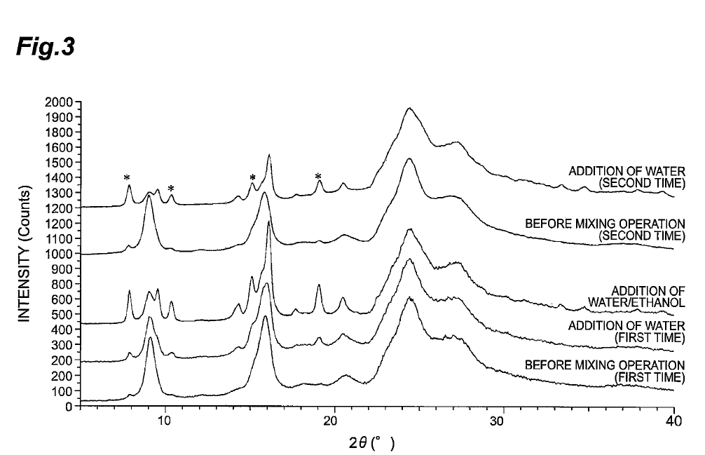

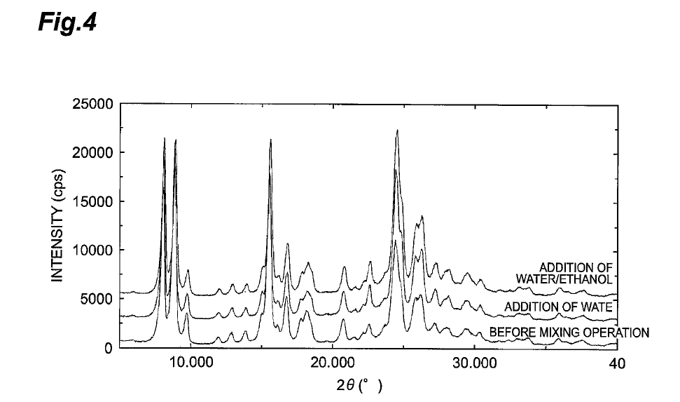

The powder X-ray diffraction pattern of Anhydrous form II before and after mixing operation is shown in FIG. 3, and the powder X-ray diffraction pattern of Crystal form IV is shown in FIG. 4. The mixing operation of Anhydrous form II in the presence of water was performed twice. The powder X-ray diffraction pattern of resulting crystals is also shown in FIG. 3.

### (Results)

Based on the powder X-ray diffraction, it can be found that the crystals obtained in Reference Example A1 (Anhydrous form II) change their crystal forms in mixing operation in the presence of water or water-ethanol (1:1) mixed solution, and crystals (crystals having peaks shown by asterisk marks in FIG. 3) which are different from the crystals obtained in Reference Example A1 increase.

On the other hand, it can be found that the crystals obtained in Example 1 (Crystal form IV) do not change their crystal forms, and they are physically stable in mixing operation in the presence of water or water-ethanol (1:1) mixed solution.

Minimum Ignition Energy and Lower Explosion Concentration Limit

### (Operation Method)

An appropriate amount of Hydrate corresponding to a concentration is uniformly put on sample dish of blown-up type dust explosion test apparatus. 50 kPa of air is compressed in a 1.3 L pressure tank, and the air is introduced into a glass cylinder by opening of a solenoid operated valve to form dust clouds. A discharge electrode is supplied with energy after 0.1 seconds after the opening of the solenoid operated valve. The criterion of ignition is an arrival of flame at an ignition mark set 100 mm above the discharge electrode.

### (Measurement Conditions for Lower Explosion Concentration Limit)

Temperature of the measurement room: 24° C.

### Humidity: 49%

Popping pressure of compressed air: 50 kPa
Ignition start time: 0.1 sec
Repetition of ignition test: 5 times
Ignition discharge energy: 10 J

### (Measurement Conditions for Minimum Ignition Energy)

Temperature of the measurement room: 24° C.

### Humidity: 49%

Popping pressure of compressed air: 50 kPa
Ignition start time: 0.1 sec
Repetition of ignition test: 10 times

### (Apparatus)

Blown-up type dust explosion test apparatus (Environmental Technology Co., Ltd. DES-10)

Chargeability

### (Operation Method)

About 1 g of respective compounds is weighed into a weighing bottle (diameter of 35 mm). A stirring bar [fluoroplastic (tetrafluoroethylene resin) coating; 20 mm] is placed in the bottle and after the lid is in place, the powders are stirred for 30 minutes. The lid is opened at the same time that stirring is stopped; and the static potential of the powder is measured using a static potential measuring instrument.

### (Apparatus)

STATIRON-DZ3 manufactured by Shishido Electrostatic, Ltd.

### (Results)

Crystals of Reference Example A1 (Anhydrous form II): 70-100 V

### INDUSTRIAL APPLICABILITY

The crystals of the present invention have preferable properties and are suitable for use as an active ingredient of therapeutic or prophylactic agents for neurodegenerative diseases or the like.

1. A crystal of 3-(2-cyanophenyl)-5-(2-pyridyl)-1-phenyl-1,2-dihydropyridin-2-one having a diffraction peak at a diffraction angle (2θ±0.2°) of 15.4° in a powder X-ray diffraction.

2. The crystal according to claim 1 further having diffraction peaks at diffraction angles (2θ±0.2°) of 16.6° and 24.3° in a powder X-ray diffraction.

3. A medicament comprising the crystal according to claim 1.

4. A pharmaceutical composition comprising the crystal according to claim 1.
